(19) United States
(12) Patent Application Publication
(10) Pub. No.: US 2024/0091087 A1
(43) Pub. Date: Mar. 21, 2024

(54) Mobility-Assistive Garment

(71) Applicant: Encore Innovation Group LLC, Livonia, MI (US)

(72) Inventors: Michael James Mies, Plymouth, MI (US); Waldemar Tadeusz Raczkowski, Farmington Hills, MI (US); Wiliam Francis Hayden, Dearborn Heights, MI (US)

(21) Appl. No.: 18/275,661

(22) PCT Filed: Jan. 17, 2022
(86) PCT No.: PCT/US2022/012657

§ 371 Date: Aug. 3, 2023

Related U.S. Application Data
(60) Provisional application No. 63/199,948, filed on Feb. 4, 2021.

(51) Int. Cl. A61G 7/10; A41D 13/00; A41D 13/12

(52) U.S. Cl. CPC A61G 7/1051; A41D 13/0007; A41D 13/1254

(57) ABSTRACT

A garment includes an interior surface, a strap sheath carried by the interior surface and establishing a strap tunnel, a strap movably extending through the strap sheath and being slidable in the strap tunnel relative to the strap sheath. Lower and upper body versions of the garment are also disclosed, as is a method of repositioning an appendage of a wearer of a garment.

### TECHNICAL FIELD

This disclosure relates generally to wearing apparel and, more particularly, to a medical device garment for assisting a wearer with mobility.

### BACKGROUND

Typically, when a paralyzed individual with limited lower body mobility wishes to reposition their lower appendages, they must use a specialized piece of equipment such as a foot lifter. These devices are usually bulky and not easily carriable by the user. Additionally, such appendage repositioning aids are costly, complicated, cumbersome, and unsightly in that they do not blend with the everyday clothing an individual may wear. Moreover, it is not uncommon for a paralyzed individual to require multiple transfers during a day. A transfer typically involves an individual repositioning their lower appendages from a first position to a second position, then pivoting their entire body to the desired second position. When an individual is in a hospital or care facility, assistants are sometimes required to assist with transfers. In such instances, the assistant may be required to intrusively place an assistive device around the appendage of a paralyzed individual. When no assistive device is available, the assistant or paralyzed individual may be required to grasp a piece of clothing or their appendage directly in order to reposition. This can result in discomfort or injury to the individual.

### BRIEF DESCRIPTION OF THE DRAWINGS

FIG. 1 is a front perspective view showing an illustrative embodiment of a mobility-assistive garment, in the form of pants, worn by a wearer in a wheelchair;

FIG. 2 is a side view of the garment of FIG. 1, showing a portion of a strap of the garment in an exposed position;

FIG. 2A is side view of the garment showing the strap in a stored position;

FIG. 3 is a front view of the garment showing the portion of the strap in the exposed position;

FIG. 3A is a front view of the garment showing the strap in the stored position;

FIG. 4 is a fragmentary, longitudinal cross-sectional view showing a structural relationship of the garment, between the strap, a strap sheath, and a pant pocket;

FIG. 5 is a front view according to another illustrative embodiment of a mobility-assistive garment;

FIG. 6 is a side view according to the garment of FIG. 5;

FIG. 7 is a front view according to an additional illustrative embodiment of a mobility-assistive garment;

FIG. 8 is a rear view of the garment of FIG. 7;

FIG. 9 is an internal view of an inside surface of the garment of FIG. 7, generally corresponding to circle 9 thereof; and

FIGS. 10 and 10A are fragmentary views of the garment of FIG. 8 corresponding to arrow 10 thereof.

### SUMMARY

In an embodiment of the present disclosure, a garment includes an interior surface, a strap sheath carried by the interior surface and establishing a strap tunnel, a strap movably extending through the strap sheath and being slidable in the strap tunnel relative to the strap sheath. Lower and upper body versions of the garment are also disclosed.

In another embodiment of the present disclosure, a method of repositioning an appendage of a wearer of a garment is disclosed. The method includes: coupling an appendage of the wearer to an appendage coupling of a slidable strap of a garment worn by the wearer; pulling on a handle of the slidable strap of the garment, so as to lift the wearer's appendage and slide the slidable strap inside of a strap tunnel established by a sheath coupled to an interior surface of an appendage tunnel of the garment and extending along the appendage tunnel; swinging an appendage of the wearer of the garment from a first position to a second position, while carrying out the pulling step; and releasing the slidable strap once the appendage has been swung to the second position.

### DETAILED DESCRIPTION

In general, a mobility-assistive garment will be described using one or more examples of illustrative embodiments of a garment with an assistive mobility feature. In one example, the garment includes a strap having a handle and an appendage coupling for lifting and moving an appendage. In another example, the garment includes a torso strap. The example embodiment(s) will be described with reference to pants and a sweatshirt for use by a paralyzed individual. However, it will be appreciated as the description proceeds that the disclosed subject matter is useful in many different applications and may be implemented in many other embodiments. In any case, the presently disclosed mobility-assistive garment may be considered a medical device, essential for providing professional care to a wearer in an effective and dignified manner.

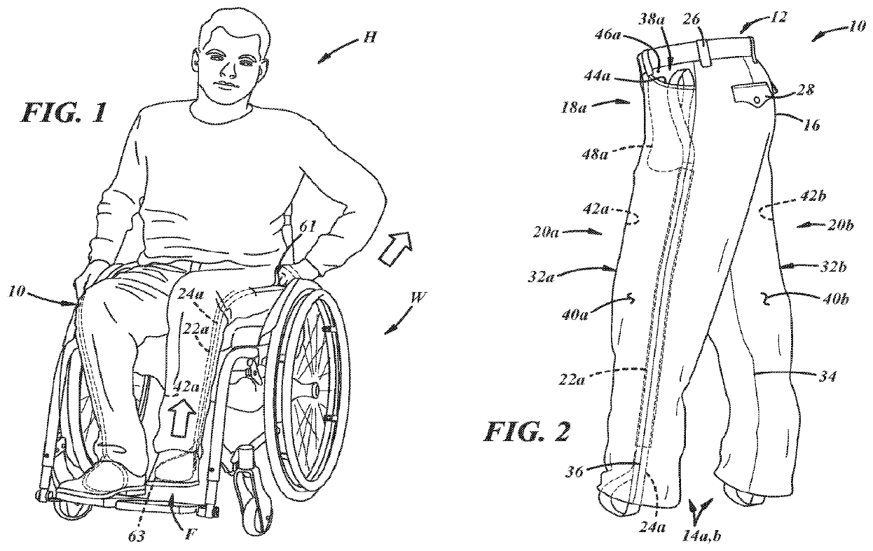

Referring specifically to the drawings, FIG. 1 shows a human wearer H of clothing sitting in a wheelchair W and wearing an article of clothing or a garment according to an illustrative embodiment of the present disclosure. The wearer H is depicted using the garment 10 to lift the wearer's foot F as will be described in greater detail below.

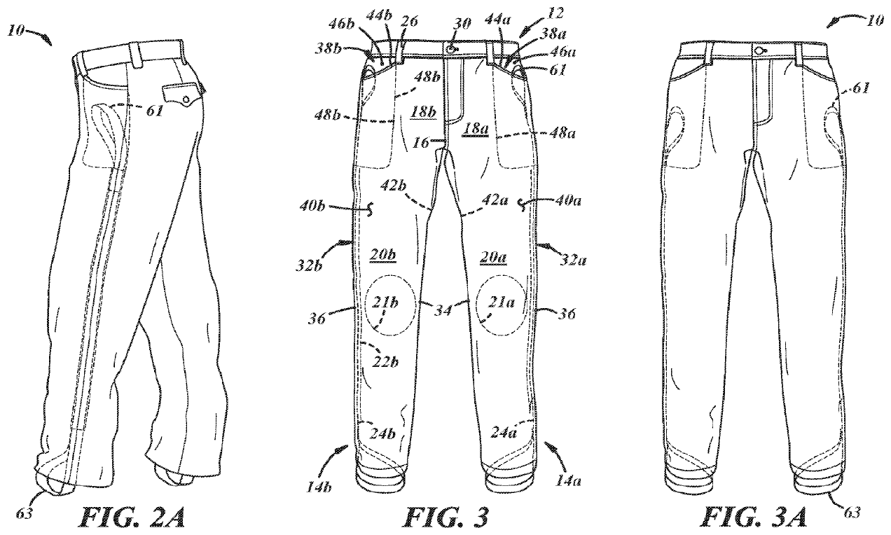

With reference to FIGS. 2 and 3, the illustrative garment 10 is a lower body garment and may include a waist band 12 to receive a user's waist, pant legs or legs 14a,b extending from the waist band 12, connected by a crotch seam 16 extending along a direction from a front of the waist band 12 toward a rear of the waist band 12. The legs 14a,b include upper portions 18a,b to receive a lower portion of the wearer's torso and lower portions 20a,b extending away from the upper portions 18a,b to receive the wearer's legs. The garment 10 also includes strap sheaths 22a,b carried by the legs 14a,b, and straps 24a,b movably extending through the strap sheaths 22a,b. The garment 10 also may include belt loops 26, rear pockets 28, buttons 30, and/or any other garment accessories.

In general, the garment 10 may be made of a fabric, other woven material, or any material whatsoever that is suitable for use as a garment for a human. A suitable fabric may include one that is resistant to tearing or stretching during use. For example, garment may be made of a strong heavy fabric, such as, for example, rip-stop nylon, polyester, or the like. Another suitable fabric may include cotton. A blended fabric may also be used such as, for example, a cotton-polyester blend. For example, a cotton-polyester blend having at least 2% polyester by weight may be used. Fabrication from a cotton-polyester blend may enhance the comfort when worn. A cotton-polyester blend may also be more resilient during use than pure cotton. A cotton-polyester blend may also be machine washable and/or suitable for machine drying.

Also, in the illustrated embodiment, the garment 10 may be outerwear including pants. As used herein, the term “pants” includes trousers, breeches, slacks, knickerbockers, britches, dungarees, jeans, knickers, pantaloons, sweatpants, or the like or any other leg covering garment that includes leg tunnels configured to extend beyond a wearer's knees. In other embodiments, a garment according to the present disclosure may include an upper torso garment, for example, a sweatshirt, sweater, shirt, coat, jacket, suitcoat, sport coat, or the like, or any other such garment that includes arm tunnels.

With reference to FIGS. 2 and 3, the lower portions 20a,b of the legs 14a,b include appendage or leg tunnels 32a,b for receiving a wearer's legs, inner and outer seams 34, 36, and front pockets 38a,b. The leg tunnels 32a,b surround the wearer's appendages and include exterior surfaces 40a,b and interior surfaces 42a,b. The leg tunnels 32a,b may also include the seams 34, 36 that extend longitudinally along the length of the legs 32a,b and may extend to the waist band 12. In the illustrated embodiment, the pockets 38a,b are located near proximate ends of the legs 32a,b. However, in other embodiments, the pockets 38a,b may be located anywhere along the legs 32a,b. As used herein, the terminology “proximate” means closer to one end than another, opposite end.

With continued reference to FIGS. 2 and 3, the pockets 38a,b may be formed as traditional style pants pockets in which a wearer stores objects or places the wearer's hands. In the illustrated embodiment, the pockets 38a,b may be established by upper edges 44a,b of the upper portions 18a,b of the legs 14a,b, external panels 46a,b extending down from the waist band 12, and by internal panels 48a,b attached to the external panels 46a,b and to inside surfaces at the upper edges 44a,b. In other embodiments, the pockets 38a,b may be established by separate pouches sewn or otherwise coupled to the legs 14a,b, or any other pocket configurations suitable for use with pants.

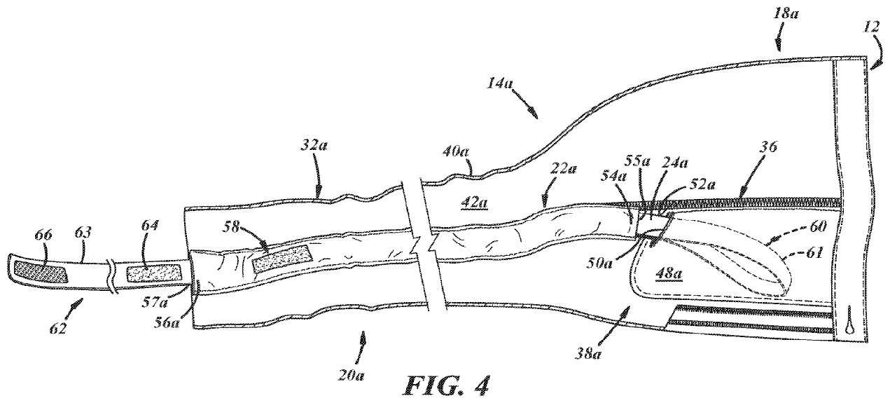

With reference to FIG. 4, the garment 10 is shown in fragmentary longitudinal cross section. As the description proceeds below, reference will be made with respect to the left leg 14a and/or portions thereof. The garment 10 includes a strap opening 50a of the pocket 38a to allow a portion of the strap 24a to pass therethrough. The opening 50a is appropriately sized to allow the strap 24a to pass through and may include a slit, a hole, a gap between an edge of pant pocket material and pant appendage material, and the like. As illustrated, the opening 50a is a slit in the internal panel 48a proximate a lower edge 52a of the pocket 38a, thereby providing a connection between the interior of the pocket 38a and the interior of the leg tunnel 32a. The interior of the leg tunnel 32a is established by the interior surface 42a of the leg tunnel 32 which circumferentially extends around a longitudinal axis of the leg tunnel 32.

With continued reference to FIG. 4, the strap sheath 22a may be carried by the interior surface 42a of the leg tunnel 32a, and may be sewn or otherwise attached to the interior surface 42a along the outer seam 36 of the leg 14a to not only create an aesthetically uniform look but also increase overall strength of the outer seam 36. More specifically, the strap sheath 22a may straddle the outer seam 36 within the interior of the leg tunnel 32a. The strap sheath 22a may be made from a material that is durable and resistant to abrasion, for example, a nylon material.

The strap sheath 22a comprises a first or upper end 54a, a second or lower end 56a, and establishes a strap tunnel between the first and second ends 54a, 56a for carrying the strap 24a. In one embodiment, the sheath 22a may include a circumferentially continuous or tubular component having an outer side coupled to the interior surface 42a of the leg tunnel 32a, for instance, sewn to the outer seam 36 of the leg 14a within the leg tunnel 32a. Accordingly, the sheath 22a may be sewn along, to, and over the outer seam 36 within the interior of the leg tunnel 32a. In another embodiment, the sheath 22a may include a planar component having laterally opposite edges coupled to the interior surface 42a of the leg tunnel 32a, for example, sewn to the outer seam 36 of the leg 14a within the leg tunnel 32a. At the upper end 54a, the strap sheath 22a includes an upper opening 55a for receiving the strap 24a, wherein the upper opening 55a may be located beneath the opening 50a of the pocket 38a. At the lower end 56a, the sheath 22a includes a lower opening 57a that may be located above the bottom of the leg tunnel 32a of the leg 14a, for example, at or above a hem thereof. A strap fastener 58 may be provided on the sheath 22a proximate the lower opening 57a, for example, within ten inches thereof. The fastener 58 may be part of a hook and loop fastener or variations thereof (Dual-Lock, mushroom stems, etc.), a button, or a button hole, magnet, or any other suitable fastener.

The sheath 22a may carry the strap 24a along a majority (greater than 50%) of the length of the leg tunnel 32a of the leg 14a. Accordingly, the sheath 22a may extend along a majority of the length of the leg tunnel 32a. More specifically, the sheath 22a may extend from a location below a knee portion (see, e.g., FIG. 3; 21a,b) of the leg 14a to a location above the knee portion. The knee portion is that portion of the leg 14a that corresponds to the location of a wearer's knee when the wearer is wearing the garment 10. Even more specifically, the sheath 22 may extend from a location within zero to three inches (including all ranges, sub-ranges, and values within that range) of the distal end of the leg 14a to a location above the knee portion of the leg 14a and, more particularly, to a location within zero to three inches (including all ranges, sub-ranges, and values within that range) of the pocket 38a.

With continued reference to FIG. 4, in the illustrated embodiment, the strap 24a includes a proximate end 60 that may be configured to extend into the pocket 38a and a distal end 62 that may be configured to extend out of the distal end of the leg tunnel 32a. The proximate end 60 of the strap 24a may include a grip or handle 61 to be gripped by a hand of the wearer or an assistant. In the illustrated embodiment, the handle 61 is formed as a loop by stitching a portion of the strap 24a to itself but, in other embodiments, the handle 61 may be formed by other means, such as, for example, a straight end of the strap 24a or a separate handle of any suitable shape sewn, tied, adhered, or otherwise coupled to the strap 24a. When not in use, the handle 61 may be stored within the pocket 38a, thereby creating an aesthetically uniform look to the garment 10, as shown in FIGS. 2A and 3A. When the wearer desires to use the handle 61, it may be retrieved from the pocket 38a and pulled out as shown in FIGS. 2 and 3.

The distal end 62 of the strap 24a may include a foot coupling 63 for receiving a wearer's foot. As used herein, the term “foot” includes a wearer's bare foot, sock-covered foot, footwear-covered foot, and the like. The foot coupling 63 may be a foot loop for receiving a wearer's foot, or footwear integrated with the strap 24a, or any other coupling suitable for use in coupling a foot to the strap 24a. As illustrated, the foot coupling 63 is shown in an open state but may be closed into loop form by attaching a portion of the distal end 62 to itself, for example, via hook and loop fasteners or variations thereof (Dual-Lock, mushroom stems, etc.), a button, or a button hole, magnet, or any other suitable fastener. More specifically, the foot coupling 63 may be formed at the distal end of the strap by fastening a first strap fastener 64 to a second strap fastener 66. The fasteners 64, 66 may be coupled to the strap 24a in such a position relative to one another so as to create loop sized for a wearer's foot when the fasteners 64, 66 are fastened together. When the foot coupling 63 is not in use, one or both of the fasteners 64, 66 may be fastened to the strap fastener 58, which may be located on the sheath 22a or may be located on the interior surface 42a of the leg tunnel 32a, to thereby conceal the strap 24a within the leg tunnel 32a. In other embodiments, a foot coupling may be established by sewing a portion of the strap 24a onto itself, or by attaching a separate handle to the strap 24a.

With continued reference to FIG. 4, the strap 24a extends along the length of the leg tunnel 32a through the strap tunnel of the sheath 22a and extends a distance greater than the length of the sheath 22a. At the proximate end of the strap sheath 22a, the strap 24a exits from the upper opening and continues into the pocket 38a through the opening 50a. The handle 61 is therefore accessible by the wearer or an assistant. When a pulling force is applied to the handle 61, the strap 24a may freely slide through the strap tunnel established by the sheath 22a alone or by the sheath 22a and the leg 14a. The strap 24a is preferably fabricated from, for example, a heavy-duty nylon, a polypropylene, or any other durable flexible material that is resistant to tearing. Further, the strap 24a may be dimensioned so as to be usable for specific clothing sizes, i.e., a shorter strap for a size “small” piece of clothing, and the like.

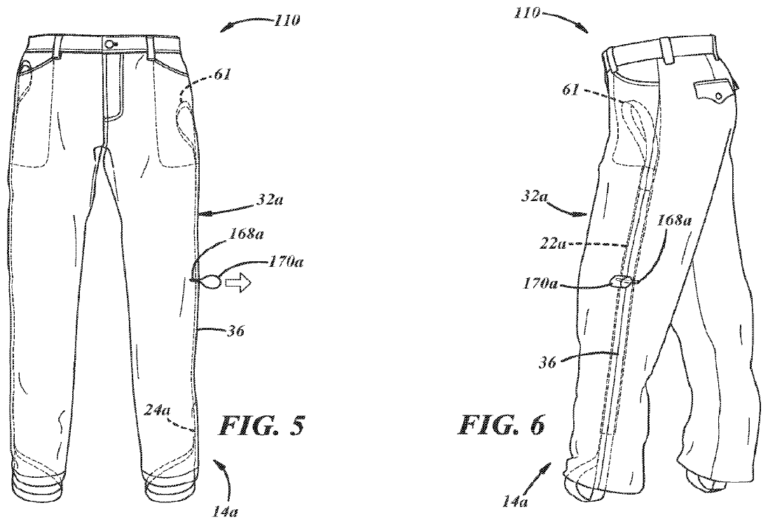

FIGS. 5 and 6 show another illustrative embodiment of a garment 110 in which the strap 24a may protrude from a leg 14a nearer a knee portion of the leg 14a, as will be described in greater detail herein below. This embodiment is similar in many respects to the embodiment of FIGS. 1-4 and like numerals between the embodiments generally designate like or corresponding elements throughout the several views of the drawing figures. Accordingly, the descriptions of the embodiments are hereby incorporated into one another, and description of subject matter common to the embodiments generally may not be repeated.

In this embodiment, the leg tunnel 32a of the leg 14a includes an opening 168a in a middle one-third of the length of the leg tunnel 32a in a location of the leg tunnel 132a proximate to a wearer's knee, to permit access to the strap 24a. The opening 168a may be in an outer side of the leg tunnel 32a, for example, traversing through the outer seam 36 as illustrated. The opening 168a may be a simple slit through the material of the leg tunnel 32a, or a hemmed opening like a button hole, or a small pocket, or any other suitable opening. In other embodiments, the opening 150a may be circumferentially spaced away from the seam 36, for example, adjacent to the outer seam 36 toward a front surface of the leg tunnel 32a. In any case, although not illustrated, a sheath opening corresponding to the leg tunnel opening 168a may be provided through the sheath 22a to permit access to the strap 24a therethrough. The strap 24a includes an intermediate handle 170a that may be created when a user reaches the user's fingers through the opening 168a and through the corresponding sheath opening and pulls a loop portion of the strap 24a therethrough and out of the leg 14a to create the intermediate handle 170a. In other embodiments, the strap 24a need not include the upper handle 61 and, instead, an upper portion of the strap 24a could be fixed to a portion of the garment 10, for instance, the leg 14a. In such embodiments, the intermediate handle 170a would serve as the only handle. Those of ordinary skill in the art would recognize that the features of this embodiment could be provided to one or the other of the legs 14a,b or to both.

Referring again to FIG. 1, also disclosed is method of repositioning an appendage of the wearer W of the garment 10. The method includes coupling an appendage F of the wearer W to an appendage coupling 63 of a slidable strap 24a of the garment 10 worn by the wearer W of the garment 10. The method also includes pulling on a handle 61, 170a of the slidable strap 24a so as to lift the wearer's appendage and slide the slidable strap 24a inside of a strap tunnel established by a sheath 22a coupled to an interior surface 42a of an appendage tunnel 32a of the garment 10 and extending along the appendage tunnel 32a. The method additionally includes swinging an appendage F of the wearer W of the garment 10 from a first position to a second position, while carrying out the pulling step. The method further includes releasing the slidable strap 24a once the appendage F has been swung to the second position. This method may be used by the individual wearing garment and/or by an individual assisting the wearer.

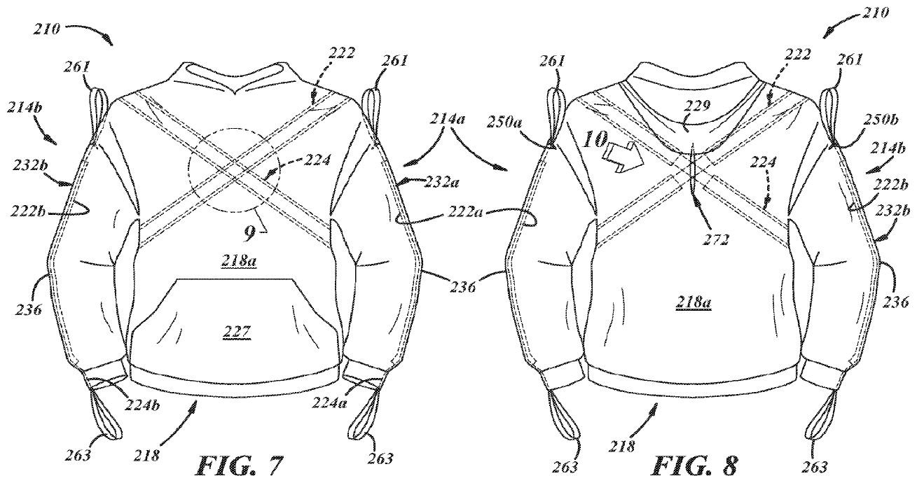

FIGS. 7 and 8 show an additional illustrative embodiment of a garment 210 to assist a wearer with mobility, as will be described in greater detail herein below. This embodiment is similar in many respects to the embodiment of FIGS. 1-6 and like numerals between the embodiments generally designate like or corresponding elements throughout the several views of the drawing figures. Accordingly, the descriptions of the embodiments are hereby incorporated into one another, and description of subject matter common to the embodiments generally may not be repeated.

In this illustrative embodiment, the garment 210 is an upper body garment and, more specifically, a sweatshirt. But, in other embodiments, the garment 210 could be a sweater, shirt, coat, jacket, suitcoat, sport coat, or the like, or any other upper body garment that includes arm tunnels. In the illustrated embodiment, the garment 210 may include a waist band 212 to receive a user's waist, a torso portion 218 to receive a user's torso and including a front torso portion 218a and a rear torso portion 218b, and sleeves 214a,b extending from the torso portion 218. The garment 210 also includes strap sheaths 222a,b carried by the sleeves 214a,b, and straps 224a,b movably extending through the strap sheaths 222a,b. The garment 210 also may include pockets 227, a hood 229, and/or any other garment accessories. Arm tunnels 232a,b may also include outer seams 236 that extend longitudinally along the length of the arm tunnels 232a,b.

With reference to FIG. 8, the garment 210 includes strap openings 250a,b to allow portions of the straps 224a,b to pass therethrough. The openings 250a,b are appropriately sized to allow the straps 224a to pass through and may include slits, holes, gaps between material portions, and/or the like. As illustrated, the openings 250a,b are slits in the arm tunnels 232a,b proximate a shoulder end of the arm tunnels 232a,b, and on a rearward facing surface of the arm tunnels 232a,b. Like the pants embodiments, and although not shown here, the garment 210 could include pockets in the region of the openings 250a,b to hide the handles 261 when not in use. Likewise, like the second pants embodiment, and although not shown here, the garment 210 could include intermediate handles and corresponding openings proximate an elbow portion of the sleeves 214a,b that corresponds to the location of a wearer's elbow when the wearer is wearing the garment 210. For example, such handles and openings could be provided in a middle one-third of the length of the sleeves 214a,b.

With reference to FIGS. 7 and 8, the garment 210 also may include assistive torso mobility features. For example, the garment 210 may include a strap 224 that may cross over the front torso portion 218a of the garment 210 as shown in FIG. 7 and the rear torso portion 218b of the garment 210 as shown in FIG. 8. In some embodiments, the strap 224 may be one continuous strap that crisscrosses over itself, is configured to extend in a FIG. 8 pattern over the torso, and that terminates in opposed ends that are sewn, tied, adhered, or otherwise coupled to one another. In the illustrated embodiment, the strap 224 may be constituted from two more separate strap portions that may be independently slidable or may be sewn, tied, adhered, or otherwise coupled to one another, for instance, at the rear torso portion 218a of the garment 210. In any case, the strap 224 preferably extends across/over shoulder portions of the garment 210 and across/under armpit portions of the garment 210, and preferably is carried in a sheath 222 that is sewn, adhered, or otherwise coupled to an inside surface of a torso portion of the garment 210. As illustrated, the sheath 222 may be constituted from two separate sheath portions having opposed ends at the rear torso portion 218a. In other embodiments, the sheath 222 may be one continuous sheath that crisscrosses over itself with suitable openings therein for the strap 224 to exit, or may be constituted from more than two separate sheath portions.

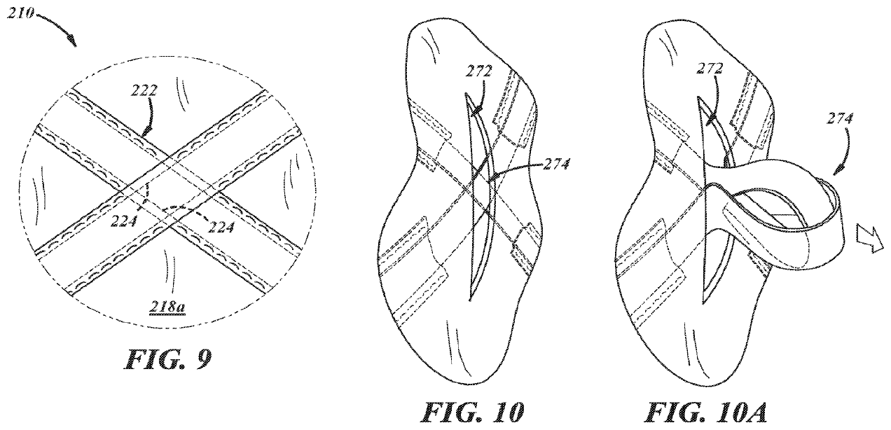

With reference to FIG. 9, an underlying portion of the crisscrossed sheath 222 may be sewn, adhered, or otherwise coupled to an inside surface of front torso portion 218a of the garment 210 and, similarly, an overlying portion of the crisscrossed sheath 222 may be sewn, adhered, or otherwise coupled to the inside surface of the front torso portion 218a of the garment 210 but not to the underlying portion of the sheath 222. Accordingly, crisscrossed portions of the strap 224 carried in the sheath 222 may slide relative to one another within the sheath 222 in which the strap 224 is slidably carried. In other embodiments, the sheath 222 may be interrupted with spaced apart ends where the strap 224 crosses over itself at the front torso portion 218a such that the strap 224 crisscrosses itself in direct contact with itself.

With reference to FIG. 10, a crisscrossed portion of the strap 224 may be accessed by hand via a vent, slit, or other opening 272 in a rear torso portion of the garment 210. In the illustrated embodiment, the opening 272 is a vertically oriented slit. But, in other embodiments, an opening could be horizontally oriented, obliquely oriented, equipped with a zipper, hook-and-loop fastener, button(s), snaps, or any other coupler suitable for use with clothing to close and open the opening. With reference to FIG. 10A, the crisscrossed portion of the strap 224 may be pulled out of the opening 272 by hand to establish a handle 274 of the strap 224 that may be used to constrict the strap 224 around the torso of the wearer of the garment 210 to assist with lifting or otherwise moving the wearer. The handle 274 may be constituted by both underlying and overlying portions of the strap 224 as illustrated. In other embodiments, the handle 274 may be constituted by only the overlying portion of the strap 224. In either case, pulling on the handle 274 slidably constricts the strap 224 around the torso of the wearer and facilitates assisting the wearer with mobility.

Prior approaches to use of assistive mobility devices involve complicated, costly, and cumbersome configurations. In contrast, the present disclosure provides a simpler, more economical, and streamlined configuration.

As used in this patent application, the terminology “for example,” “for instance,” “like,” “such as,” “comprising,” “having,” “including,” and the like, when used with a listing of one or more elements, is open-ended, meaning that the listing does not exclude additional elements. Likewise, when preceding an element, the articles “a,” “an,” “the,” and “said” mean that there are one or more of the elements. Moreover, directional words such as front, rear, top, bottom, upper, lower, radial, circumferential, axial, lateral, longitudinal, vertical, horizontal, transverse, and/or the like are employed by way of example and not limitation. As used herein, the term “may” is an expedient merely to indicate optionality, for instance, of an element, feature, or other thing, and cannot be reasonably construed as rendering indefinite any disclosure herein. Other terms are to be interpreted and construed in the broadest reasonable manner in accordance with their ordinary and customary meaning in the art, unless the terms are used in a context that requires a different interpretation.

Finally, the present disclosure is not a definitive presentation of an invention claimed in this patent application, but is merely a presentation of examples of illustrative embodiments of the claimed invention. More specifically, the present disclosure sets forth one or more examples that are not limitations on the scope of the claimed invention or on terminology used in the accompanying claims, except where terminology is expressly defined herein. And although the present disclosure sets forth a limited number of examples, many other examples may exist now or are yet to be discovered and, thus, it is neither intended nor possible to disclose all possible manifestations of the claimed invention. In fact, various equivalents will become apparent to artisans of ordinary skill in view of the present disclosure and will fall within the spirit and broad scope of the accompanying claims. Features of various implementing embodiments may be combined to form further embodiments of the invention. Therefore, the claimed invention is not limited to the particular examples of illustrative embodiments disclosed herein but, instead, is defined by the accompanying claims.

1. A garment, comprising:
an interior surface;
a strap sheath carried by the interior surface and establishing a strap tunnel; and
a strap movably extending through the strap sheath and being slidable in the strap tunnel relative to the strap sheath.

2. The garment of claim 1, wherein:
the interior surface is in an appendage tunnel of the garment;
the strap sheath extends along the appendage tunnel; and
the strap extends a distance greater than a length of the strap sheath and is slidable relative to the appendage tunnel and the strap sheath.

3. The garment of claim 2, wherein the appendage tunnel is a sleeve or a leg.

4. The garment of claim 2, wherein the appendage tunnel also has a longitudinally extending seam, and wherein the strap sheath is sewn along the seam and over the seam.

5. The garment of claim 2, wherein the strap extends between the strap sheath and the interior surface of the appendage tunnel.

6. The garment of claim 2, wherein the strap sheath is a tubular component establishing the strap tunnel and the strap extends through the strap sheath.

7. The garment of claim 2, wherein the strap has a distal end including a foot or handle.

8. The garment of claim 7, wherein the foot or handle includes a strap fastener and the strap sheath includes a sheath fastener to fasten to the strap fastener.

9. The garment of claim 2, wherein the strap has a proximate end including a handle.

10. The garment of claim 2, wherein the strap sheath extends along a majority of a length of the appendage tunnel.

11. The garment of claim 2, wherein the appendage tunnel includes a pocket, and wherein the strap extends through an opening of the pocket.

12. The garment of claim 2, wherein the appendage tunnel includes an opening in a middle one-third of the length of the appendage tunnel, and wherein the strap is extendible through the opening of the appendage tunnel to form an intermediate handle.

13. The garment of claim 12, wherein the opening is in a knee or elbow region of the appendage tunnel.

14. A lower body garment, comprising:
a leg having an interior surface, a longitudinally extending seam, and a pocket;
a strap sheath sewn along, to, and over the seam of the leg, and extending along the appendage tunnel over a majority of a length of the leg, and establishing a strap tunnel; and
a strap extending through the strap sheath and into the pocket of the leg, and extending a distance greater than the length of the strap sheath and being slidable in the strap tunnel relative to the leg and the strap sheath, and having a distal end including a foot coupling and a proximate end including a handle.

15. The garment of claim 14, wherein the strap extends through a side of the pocket into the interior of the pocket.

16. An upper body garment, comprising:
a sleeve having an interior surface and a longitudinally extending seam;
a strap sheath sewn along, to, and over the seam of the sleeve, and extending along the sleeve over a majority of a length of the sleeve, and establishing a strap tunnel; and
a strap extending through the strap sheath and extending a distance greater than the length of the strap sheath and being slidable in the strap tunnel relative to the sleeve and the strap sheath, and having a distal end including a hand coupling and a proximate end including a handle.

17. The garment of claim 16, wherein the strap extends through an opening at a rear upper portion of the sleeve.

18. An upper body garment, comprising:
a torso portion having a front torso portion and a rear torso portion;
sleeves extending away from the torso portion;
a strap sheath being carried by the front and rear torso portions, crisscrossing over and under the sleeves, and establishing a strap tunnel; and
a strap extending through the strap sheath and extending a distance greater than the length of the strap sheath and being slidable in the strap tunnel relative to the strap sheath, and having a handle.

19. The upper body garment of claim 18, wherein the rear torso portion has an opening through which the handle of the strap is accessible.

20. A method of repositioning an appendage of a wearer of a garment, comprising:
coupling an appendage of the wearer to an appendage coupling of a slidable strap of a garment worn by the wearer;
pulling on a handle of the slidable strap of the garment, so as to lift the wearer's appendage and slide the slidable strap inside of a strap tunnel established by a sheath coupled to an interior surface of an appendage tunnel of the garment and extending along the appendage tunnel;
swinging an appendage of the wearer of the garment from a first position to a second position, while carrying out the pulling step; and
releasing the slidable strap once the appendage has been swung to the second position.
